(19) United States
(12) Patent Application Publication
(10) Pub. No.: US 2004/0013702 A1
(43) Pub. Date: Jan. 22, 2004

(54) Implantable devices for the controlled release of cytotoxic agents

(76) Inventor: Eugene G. Glover, Santa Barbara, CA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/196,463

(22) Filed: Jul. 16, 2002

(51) Int. Cl. A61K 9/22

(52) U.S. Cl. 424/423; 604/890.1

(57) ABSTRACT

An implantable device that includes at least one sealed chamber containing any of a variety of therapeutic or cytotoxic agents is described. The device is implanted into the patient at one or more sited requiring treatment, e.g., at several sites within a tumor. Once the device is implanted in the patient, the chamber retains its contents until a bioresorbable element of the chamber (e.g., a seal) dissolves, creating an opening through which the agent migrates out of the chamber and enters the body. The bioresorbable element can be simply a plug seal in a plastic or metal chamber. Alternatively, all or part of the chamber itself can be formed of a bioresorbable material. The agent can be chemical or biological.

### BACKGROUND OF THE INVENTION

Various implantable drug delivery systems designed to achieve controlled release of a therapeutic agent are known in the art. Many such systems are designed to maintain a constant, systemic therapeutic drug level over an extended period of time. For example, U.S. Pat. No. 6,126,965 describes an implantable, biocompatible biodegradable polymer that is said to continuously deliver a stable concentration of an opioid analgesic subcutaneously for periods ranging from two weeks to more than six months. Such controlled (or sustained) release drug delivery systems are designed to maintain a constant systemic concentration of a drug, thereby avoiding the peaks and valleys inherent with oral medication or intra-muscular injection.

Some implantable drug delivery systems are designed to achieve a high, relatively constant local concentration of a drug over a relatively extended period of time. Controlled release biocompatible polymers for local drug delivery have been used for contraception, glaucoma treatment, asthma therapy and chemotherapy of cancer (Langer and Wise, eds., Medical Applications of Controlled Release, Vol. I and II, CRC Press, Boca Raton, Fla. (1986)).

Controlled release devices for delivery of chemotherapeutic agents to solid tumors (See, e.g., U.S. Pat. Nos. 5,846,565 and RE 37,410) and for delivery of a pain killer (U.S. Pat. No. 6,126,956) are known in the art. Delayed release devices relying on the use of swelling agents (U.S. Pat. No. 5,654,009) or degradable microspheres (U.S. Pat. No. 5,993,885) are also known in the art. One common treatment for prostate cancer, breast cancer and other cancers in which the cancer remains encapsulated, entails placing a number of capsules (brachytherapy seeds) containing a small amount of radioactive material in and around the tumor. The brachytherapy seeds are left within the patient for a period of time to expose the tumor to killing radiation without significantly exposing surrounding healthy tissue to killing radiation.

### BRIEF DESCRIPTION OF THE DRAWINGS

FIG. 1 depicts a cross-section of an implantable capsule having a biodegradable seal at one end.

FIG. 2 depicts a cross-section of an implantable capsule having a biodegradable seal at each end.

FIG. 3 depicts a cross-section of an implantable capsule formed of biodegradable material.

FIG. 4 depicts a cross-section of an implantable capsule having two chambers each of which is sealed with a biodegradable seal.

### SUMMARY OF THE INVENTION

The invention features a device that when implanted into a patient can deliver a direct dose of therapeutic or cytotoxic agent to a localized area of the body without subjecting the patient to systemic exposure to the therapeutic or cytotoxic agent. The implantable device includes at least one sealed chamber containing any of a variety of therapeutic or cytotoxic agents. The device is implanted into the patient at one or more sited requiring treatment, e.g., at several sites within a tumor. Once the device is implanted in the patient, the chamber retains its contents until a bioresorbable element of the chamber (e.g., a seal) dissolves, creating an opening through which the agent migrates out of the chamber and enters the body. The bioresorbable element can be simply a plug seal in a plastic or metal chamber. Alternatively, all or part of the chamber itself can be formed of a bioresorbable material. The agent can be chemical or biological.

Such a device could be useful in the treatment of encapsulated cancers, as with brachytherapy seeds or in the treatment of Benign Prostatic Hypertrophy (BPH) or swelling of the prostate. The device can be filled with a cytotoxic agent, implanted into the prostate where it would remain for a time and then release the agent into the area surrounding the device causing the destruction of a small amount of tissue.

In one embodiment, the invention features an implantable device for localized delivery of an agent to a region of the body, the device comprising: (a) a chamber having one or more sealable openings and (b) a therapeutic or cytotoxic agent contained within the chamber, wherein the sealable openings are releasably sealed with a biodegradable polymer that retains the therapeutic or cytotoxic agent within the chamber for at least a first predetermined time period after implantation. In various embodiments the first predetermined period of time is more than one day but less than 100 days, less than 90 days, less than 80 days, less than 70 days, less than 60 days, less than 50 days, less than 40 days, less than 30 days, less than 20 days, less than 10 days, and less than five days.

Once the first predetermined period of time has elapsed, essentially all of the agent is free to come into contact with surrounding tissue (although it must migrate out of the chamber to do so).

In various embodiments the chamber is formed of a biodegradable material that does not substantially degrade within the first predetermined time period after implantation, the chamber is formed of a metal, the chamber is formed of a non-biodegradable material, the biodegradable polymer degrades by bulk erosion, the biodegradable polymer degrades by surface erosion, the chamber is formed of silicone rubber, and the devise further comprises a biocompatible coating on the outer surface of the chamber. The chamber can be any suitable shape including cylindrical and ovoid.

The device can include a plurality of chambers that are linked together. The chambers can differ in size, shape and other properties.

In one embodiment, the implantable device comprises at least a first chamber having at least one opening releasably sealed with a biodegradable polymer that retains the cytotoxic agent within the first chamber for at least a first predetenmined time period after implantation and at least a second chamber releasably sealed with a biodegradable polymer that retains the cytotoxic agent within the second chamber for a time period that is greater than the first predetermined time period after implantation.

The implantable device can further comprise a therapeutic or cytotoxic agent admixed with a biodegradable polymer or otherwise formulated for controlled release.

### DETAILED DESCRIPTION OF THE INVENTION

The present invention features implantable devices containing a chemically active, non-selective cytotoxic agent (e.g., an acid, base, or salt) or selective cytotoxic agent that specifically targets particular cellular factor (e.g., the topoisomerase I inhibitor camptothecin) or a therapeutic agent. The implantable devices include one or more capsules or chambers that are partially or completely filled with the agent. The capsules or chambers release their therapeutic cytotoxic agent after a predetermined time period (e.g., an hour, a few hours, a day, a few days, a week, or several weeks) subsequent to implantation within the patient, e.g., within a prostate tumor. The release of the agent takes place and is complete, in most cases, and over a relatively short period of time such that a relatively high local concentration of the therapeutic or cytotoxic agent is achieved in the region of the implant. Thus, in some embodiments, essentially all of the agent within the chamber is free to come into contact with body tissue simultaneously (e.g., within minutes or a few hours).

The capsules have one or more sealable openings that are sealed with a biodegradable polymer that degrades and releases the cytotoxic agent from the capsules after the implant has been within the patient for at least a predetermined time period. Depending on the biodegradable polymer used, the thickness of the material and other factors, the initial release of material within the capsule does not begin until hours, days, or weeks after implantation. Under some circumstances it can be desirable to implant a variety of capsules, e.g., a first type of capsule which begins to release its contents after a first period of time (e.g., a few days) can be implanted along with a second type of capsule which begins to release its contents after a second, longer period of time (e.g., two weeks). In some circumstances it can be desirable to implant into a patient several of two, three, four or more types of capsules.

The implant can be designed to release the therapeutic cytotoxic agent in a particular direction (or directions) relative to the position of the implant. For example, the one or more capsules making up the implant can be designed to release the cytotoxic agent through an opening that is located on a particular face of the capsule. The implant is then inserted into a tumor such that opening in each capsule faces the most massive portion of the tumor.

The implantable device can include a number of separate capsules that are implanted together. The various capsules can be designed to release the same or different agents and to release their contents at approximately the same time or at different times. For example, an implant might consist of five linked capsules. The first capsule could be designed to release a cytotoxic agent approximately one day after implantation, the second capsule could be designed to release a cytotoxic agent two days after implantation, the third capsule could be designed to release a cytotoxic agent after three days, and so on. The various capsules can be connected to each other in variety of geometries so as to achieve a desired distribution pattern for the cytotoxic agent. Linking the capsules decreases the migration of the capsules within the patient's body. The links between capsules can be formed of the same material as the capsules themselves or the can be formed of a different material.

A single capsule can have two or more chambers each containing the same or different agents. The two chambers can be designed to release their contents essentially simultaneously or at different times.

The capsules will in many cases be designed to resist breakdown within the patient's body. Thus, the capsules can be formed of silicone or metal and can be removed from the patient at some point after they have released their cytotoxic agent.

In an alternative embodiment, the capsules can be wholly or partially biodegradable. Thus, a capsule can be a hollow body that is formed of a biodegradable material and is partially or wholly filled with a therapeutic or cytotoxic agent or a therapeutic or cytotoxic agent compounded or admixed with other materials, e.g., a biodegradable polymer. A portion of the wall of the hollow body can be designed to biodegrade after a specified relatively short period of time such that the cytotoxic agent is released over a very short period of time. The remainder of the capsule can biodegrade more slowly after all or substantially all of the cytotoxic agent has been released.

The capsule can be a hollow body with a biodegradable portion that permits the release of a therapeutic or cytotoxic agent that is itself combined with an agent delays the effect of the agent. For example, the hollow body is filled with a sustained release formulation of the cytotoxic agent. In addition, the capsule can contain a portion of agent that is active immediately upon release from the capsule and one or more additional portions of the same or different agent that are active only after a certain time after release from the capsule.

### EXAMPLE 1

The implantable device can include one or more metal capsules of the type used for brachytherapy seeds. The capsule (or chamber) is a hollow body having one or more releaseably sealed openings for filling of the capsule with cytotoxic agent and for subsequent release of the cytotoxic agent. The opening or openings are sealed with a synthetic or natural biodegradable polymer seal (plug) formed of polylactic acid (PLA) polyglycolic acid (PGA), PLA/PGA copolymers or the like.

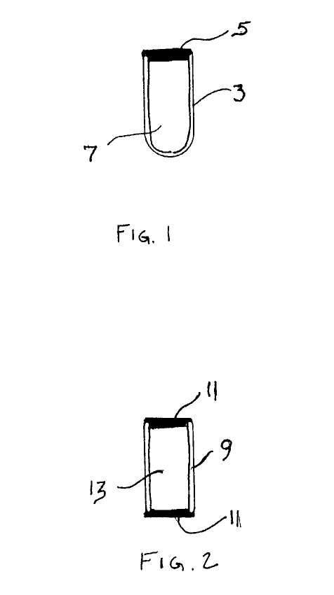

FIG. 1 depicts a cross-section of a capsule according to one embodiment of the invention. The walls 3 are metal, e.g., stainless steel, and the plug 5 at one end is formed of a biodegradable polymer. The chamber is filled with a cytotoxic agent 7.

FIG. 2 depicts a cross-section of an alternative embodiment of the invention. The capsule is cylindrical with silicone walls 9. The capsule has a biodegradable plug 11 at each end and is filled with a therapeutic agent 13.

The implantable device can implanted using, e.g., a hypodermic needle or biopsy needle as with brachytherapy techniques. The implant remains relatively inert while the insertion wound heals. Gradually, the biodegradable seal dissolves until it becomes dislodged, thereby releasing the cytotoxic agent. The cytotoxic agent is designed to be toxic to the immediately surrounding cells due to the relatively high local concentration of the agent. More remote cells and tissue would be spared and the cytotoxic agent would be tissue be safely absorbed by the body.

Suitable cytotoxic agents include salts (e.g., NaCl) acids (HCl, acetic acid), bases (NaOH), phenol, and other non-specific cytotoxic agents. Synthetic biodegradable polymers include: PLA and PGA, noted above, polyanhydrides, polyhydroxy acids and copolymers thereof, polyesters, polyamides, and polyorthoesters. Natural biodegradable polymers include proteins and polysaccharides such as collagen, hyaluronic acid, albumin and gelatin.

Many other biodegradable materials could be used to temporarily seal the sealable openings in the capsule. For example, capralactone polymers as well as polymers of dioxanone, esteramide ortremethlyenecarbonate can be used. Each of these can be copolymerized with PLA, PGA and each other.

Biodegradable polymers can degrade by surface erosion or bulk erosion. In the case of seals or plugs for temporary closing of openings in the chambers, either type can be used, although bulk eroding polymers are preferable in many circumstances. Polymers prepared from linear aliphatic diacids are hydrophilic polymers that degrade via bulk erosion.

The time required for the biodegradable portion to degrade sufficiently to release the therapeutic or toxic agent can be altered by altering the composition of the biodegradable material. For example, one can alter the ratio of p-carboxyphenoxy propane to sebacic acid, with which it is co-polymerized.

The capsule can be formed of any biocompatible, inert material, e.g., a metal (e.g., an aluminum, copper, titanium, tantalum, nickel, or stainless steel alloy), silicone rubber, polyethylene, ethylene vinyl acetate co-polymer, polyacrylonitriles, certain polyphosphazenes, and polysulfone.

An interior portion of the chamber can be threaded to receive a threaded biodegradable seal. Alternatively, the biodegradable seal can be pressured fitted. It may be desirable to roughen or texture at least the portion of the interior surface of the chamber that comes into contact with the biodegradable seal. The seal can extend or the outside surface of the device in the region surrounding the sealable opening, and it can be desirable to roughed or texture the portion of the outside surface that comes into contact with the biodegradable seal.

### EXAMPLE 2

The capsules can be formed from totally of biodegradable polymer. Such capsules can include portions that degrade more quickly and portions that degrade less quickly. For example, the thickness of the walls of the capsule can be variable to geometrically control the degree of biodegradation of the capsule. Such a device would release the enclosed cytotoxic agents in a direction dictated by the shape and composition of the capsule. Alternatively a capsule formed of a first slowly degrading biodegradable polymer can be provided with openings that are sealed with a more rapidly degrading biodegradable polymer.

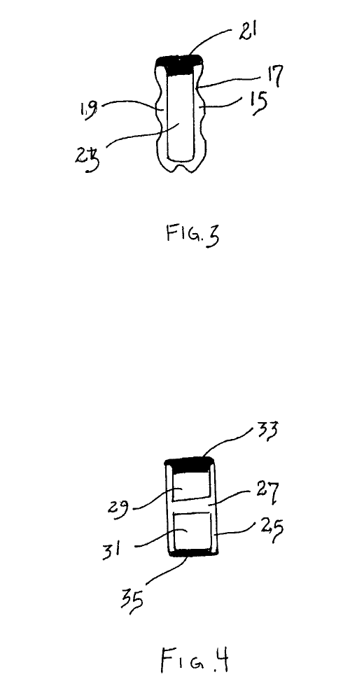

FIG. 3 depicts the cross-section of one embodiment of a capsule formed entirely of a biodegradable material. The wall 15 of the capsule varies in thickness such that there are thinner portions 17 and thicker portions 19. The capsule is sealed with a biodegradable plug 21 and is filled with a therapeutic agent 23. The thinner wall portions 17 will degrade more quickly than the thicker wall portions 19, providing openings in the capsule. The capsule can be formed using a mold, filled with therapeutic agent and then sealed with a plug.

### EXAMPLE 3

The capsule or capsules of the implantable device can contain microencapsulated cytotoxic agents. The microencapsulation is designed to achieve sustained release of the chemical cytotoxic agent. When the capsule releases the microencapsulated cytotoxic agent, release of the cytotoxic agent into the surrounding cells is initiated. This release would then occur at the rate determined by the degree or type of microencapsulation. Such an arrangement provides further time and migration control over the chemical release properties.

The cytotoxic agent can be microencapsulated using any of a variety of sustained release formulations. For example, ethylene-vinyl acetate (EVA) is commonly used to achieve sustained release of therapeutic agents. EVA is biocompatible, non-inflammatory, and largely non-biodegradable. A variety of factors including: the molecular weight, charge and lipid solubility of the agent, the charge and other characteristics of the polymer, and the percentage loading of the agent all can influence the release kinetics of the agent. Formulation of various drugs with EVA is well-known in the art (Sefton et al. (1985) J. Pharm. Sci. 73:1859; Brown et al. (1983) J. Pharm Sci. 1181; Brook et al. (1984) Br. Den. J. 157:11). Because EVA is largely non-biodegradable, the microcapsules can be removed from the patient largely intact if necessary.

### EXAMPLE 4

Cytotoxic chemical agents can be incorporated into a biodegradable polymer. In this embodiment, there is no separate hollow chamber that is filled with the cytotoxic agent. Instead the implant comprises a number of unitary bodies. As the implant device is degraded by the body, the entrapped cytotoxic agent is released in a manner similar to more conventional sustained release drug delivery. The regulating factors here would be the local concentration caused by the rate of decay of the biodegradable carrier versus the rate at which the body can absorb and clear the cytotoxic agent. This approach would most likely result in lower concentrations of active ingredients.

Whether the biodegradable polymer is synthetic or natural, the cytotoxic agent is released by diffusion, degradation of the polymer, or a combination thereof. As noted above, biodegradable polymers can degrade by bulk erosion or surface erosion. Those that dissolve by surface erosion are preferred where more linear release is required.

### EXAMPLE 5

The capsule can have multiple chambers, each of which can be filled with the same agent or with different agents. The chambers can be designed to release their contents essentially simultaneously or not. For example, it may be desirable to provide a first chamber sealed with a thicker or more slowly degrading plug and a second chamber sealed with a thinner or more quickly degrading plug.

FIG. 4 depicts one embodiment of a cylindrical, multi-chamber capsule. The wall 25 of the capsule is metal, e.g., stainless steel. A dividing wall 27 bisects the cylinder to create a first chamber 29 and a second chamber 31. The first chamber is sealed with a relatively thick biodegradable plug 33 and the second chamber is sealed with a relatively thin biodegradable plug 35.

Therapeutic and Cytotoxic Agents

The devices of the invention can contain any desired therapeutic or cytotoxic agent. The amount and the concentration of the agent in a given chamber will depend on the disorder being treated, the patient being treated and other factors. The entire chamber need not be completely filled with the agent. Instead, a chamber can be partially filled. The devices of the invention are particularly useful for administration of agents that are preferably not systemically administered.

Treatment with the Device

The device of the invention is useful in any situation in which it is desirable to achieve delayed release of a therapeutic or cytotoxic agent followed by high drug loading once the agent is released. For example, the device can be used in the treatment of solid tumors to locally release a high dosage of cytotoxic or chemotherapeutic agent directly to the tumor.

The implantable devices of the invention can be introduced into a patient surgically by directly exposing the region into which they are to be implanted. Alternatively, they can be injected using a needle-like device in much the same manner that brachytherapy seeds are implanted (see, e.g., U.S. Pat. No. 5,28,130 and 6,361,487). Endoscopic cameras and ultrasound can be used to guide the placement of the devices.

Kits

Two or more devices can be provided in a kit comprising the devices and packaging to contain the devices. The kit can contain devices containing the same or different therapeutic or cytotoxic agents. The kit can package together devices containing the same or different therapeutic or cytotoxic agents and having different release times such that some of the devices in the kit release their contents after a first predetermined time and other devices in the kit release their contents after a second predetermined time. The kit can be assembled such that each device releases its contents after a different predetermined time. Thus, a kit might contain a device that releases it contents after about one week, a device that releases it contents after about two weeks, a device that releases its contents after about three weeks, and a device that releases its contents after about four weeks. The kit can include also instructions for administering or implanting the devices.

Other Embodiments

The implants of the invention can include a combination of delayed release capsules and continuous release compositions. Thus, a patient could, in a single implantation procedure, be treated with capsules that would achieve a delayed and relatively concentrated release of a cytotoxic agent and with a controlled release implant of a cytotoxic agent, e.g., a chemotherapeutic agent. Suitable chemotherapeutic agents include: doxorubicin, ternozolamide, taxol, paclitaxel, carbplatinum, and cisplatinum. For treatment of prostate cancer, suitable chemotherapeutic agents include: docetaxel, estramustine, bicalutamide and goserelin acetate.

The capsules can be formed by molding or casting. In addition, the implant can take the form of a continuous extrusion or co-extrusion such that the size or length of the implant could be under control of the surgeon. In the case of an encapsulation extrusion the surgeon could perform a simple crimping operation to reseal the cut ends of the implant.

What is claimed is:

1. An implantable device for localized delivery of an agent to a region of the body of a patient, the device comprising:
(a) at least one chamber having one or more sealable openings; and
(b) a therapeutic or a cytotoxic agent contained within the at least one chamber,
wherein the one or more sealable openings are releasably sealed with a biodegradable polymer that retains the therapeutic or cytotoxic agent within the chamber for at least a first predetermined time period after implantation of the device in a patient.

2. The implantable device of claim 1 wherein the chamber is formed of a biodegradable material that does not substantially degrade within the first predetermined time period after implantation.

3. The implantable device of claim 1 wherein the chamber is formed of a non-biodegradable material.

4. The implantable device of claim wherein the chamber is formed of a metal selected from the group consisting of a stainless steel alloy, a copper alloy, aluminum, tantalum, titanium alloy, and a nickel alloy.

5. The implantable device of claim 3 wherein the chamber is formed of silicone rubber.

6. The implantable device of claim 1 further comprising biocompatible coating on the outer surface of the device.

7. The implantable device of claim 1 further comprising a lubricious coating on the outside of the device.

8. The implantable device of claim 1 further comprising an antibiotic coating on the outside of the device.

9. The implantable device of claim 1 wherein the device is cylindrical.

10. The implantable device of claim 1 wherein the device is ovoid.

11. The implantable device of claim 1 comprising a plurality of chambers.

12. The implantable device of claim 1 comprising at least two chambers.

13. The implantable device of claim 12 comprising at least a first chamber having at least one opening releasably sealed with a biodegradable polymer that retains the therapeutic or cytotoxic agent within the first chamber for at least a first predetermined time period after implantation and at least a second chamber having at least one opening releasably sealed with a biodegradable polymer that retains the therapeutic or cytotoxic agent within the second chamber for a time after implantation that is greater than the first predetermined time period after implantation.

14. The implantable device of claim 1 wherein the therapeutic or cytotoxic agent is admixed with a biodegradable polymer.

15. The implantable device of claim 1 wherein the biodegradable polymer degrades by bulk erosion.

16. The implantable device of claim 1 wherein the biodegradable polymer degrades by surface erosion.

17. The implantable device of claim 1 having a first chamber filled with a first therapeutic or cytotoxic agent and a second chamber filled with a second, different therapeutic or cytotoxic agent.

18. The implantable device of claim 1 wherein the chamber is filled with a cytotoxic agent selected from the group consisting of: an acid, a base, and salt.

19. The implantable device of claim 1 wherein the therapeutic or cytotoxic agent is encapsulated.

20. The implantable device of claim 1 comprising a plurality of chambers, each chamber connected to at least one other chamber by a flexible linkage.

21. An implantable device for localized delivery of an agent to a region of the body, the device comprising:
(a) at least one chamber formed of a biodegradable material and having one or more sealable openings; and
(b) a therapeutic or a cytotoxic agent contained within the chamber,
wherein the sealable openings are releasably sealed with a biodegradable polymer and wherein the biodegradable polymer retains the therapeutic or cytotoxic agent within the chamber for at least a first predetermined time period after implantation.

22. The device of claim 1 wherein the predetermined period of time is more than one day, but less than 100 days.

23. The device of claim 1 wherein the predetermined period of time is more than one day, but less than 50 days.

24. The device of claim 1 wherein the predetermined period of time is more than one day, but less than 10 days.

25. A kit comprising two or more of the devices of claim 1 or claim 21 and instructions for use contained in packaging.

26. The kit of claim 25 wherein the two or more devices retain their therapeutic or cytotoxic agents for different predetermined periods of time.
